(19) United States
(12) Patent Application Publication
(10) Pub. No.: US 2003/0185811 A1
(43) Pub. Date: Oct. 2, 2003

(54) Herbal extract and preparation thereof

(76) Inventors: Steve Teasdale, Otterburn Park (CA); Corinne Lafrance, St-Amable (CA)

Correspondence Address: George A. Seaby, Seaby & Associates, 603-880 Wellington Street, Ottawa, ON, K1R 6K7, CA

(21) Appl. No.: 09/776,870

(22) Filed: Feb. 6, 2001

(51) Int. Cl. A61K 35/78; A61K 45/00; C12P 1/00

(52) U.S. Cl. 424/93.45; 424/725; 435/41

(57) ABSTRACT

A process for preparing a herbal extract including the steps of mixing herbal matter with water to produce an aqueous extract solution, adding a nutritive supplement capable of supporting bacterial fermentation to the solution, seeding the resulting mixture with probiotic bacteria, and incubating the seeded mixture to effect fermentation of the herbal matter.

### FIELD OF THE INVENTION

This invention relates to a process for producing herbal extracts.

The extracts of the present invention can be used in functional drinks and phytotherapeutic drinks and compositions containing an herbal extract, probiotic bacteria and fermented herbal extract. The probiotic bacteria are used in a traditional herbal extraction process such as infusion and decoction to generate a unique fermented herbal extract. The present process can be applied to any herbal extract that has any proven beneficial effect on health or on any herbal extract that is acceptable for human consumption.

### BACKGROUND OF THE INVENTION

Recently there has been a renewed interest in traditional therapeutic solutions to health problems. The possibility of using plants and herbal extracts to cure health disorders is being considered by increasing numbers of people. Even health professionals are more open to considering such alternative cures. The use of plants and herbal extracts for curing illness and health disorders is called phytotherapy. The science has been known and used by man for thousands of years. Until the beginning of the twentieth century, herbal extracts and plants were the major, if not the sole solutions available to cure health defects. Even without being supported by any scientific basis, the impact of herbal remedies on physiological conditions and their side effect have been well documented.

At the beginning of the twentieth century, the progress of chemistry allowed the isolation and purification of the active ingredients of therapeutic plants, thus providing better control over the dosage of active ingredients. Unfortunately, the result may be a shock to the organisms which can sometimes lead to numerous unwanted side effects. In plant tissues, the active ingredient is mixed with hundreds of natural substances. The complex mixture may have tempered and prolonged beneficial physiological effects, with limited potential side effects. As well as their potential for curing health disorders, plants and herbal extracts can sometimes provide some interesting physiological feedback to human metabolism. The most obvious example is probably the use of coffee and tea extracts to promote awareness. Recent interest in adding plants and herbal extracts to food and drinks to provide physiological feedback has lead to the creation of the term “functional” food or drink. Such food and drinks are known to promote certain physiological and health conditions.

All of the interest in the functional and therapeutic potential of plants and herbal extract has resulted in a need to create more concentrated and more effective compositions. The extraction of herbal material is well known in the art. In the following, tea extract is given as a reference model, because tea extract is the most thoroughly documented case.

Typically, green tea is extracted with water to form a dilute extract containing soluble tea components. Organic solvents can also be used to form a tea extract containing water insoluble components. Hot water is preferred because it provides faster extraction. Canadian Patent Application No. 2,176,293, filed in the name of Y. Hagiwara on May 5, 1996 describes the production of edible plant juice extracts by squeezing fresh plant material. U.S. Pat. No. 5,780,086, issued to Kirksey et al on Jul. 14, 1998 describes a tea extraction process in which organic acids such as citric acid, erythorbic acid and/or ascorbic acid are added to a water based solution to promote extraction and to stabilize the produced extract. It is known to use purified enzymes such as tannases and cellulases to enhance the coloration and concentration of tea extracted from tea leaves. U.S. Pat. No. 5,879,730 issued to Bouwens et al on Mar. 9,1999 discloses a method of enhancing the color and concentration of tea extract using exogenous lactases, tannases, polyphenol oxidase or peroxidase.

As well as a need to produce more concentrated herbal extracts there are concerns about the appearance, taste, quality and stability of such extracts. U.S. Statutory Invention Registration No. H1,628, which issued on Jan. 7, 1997 (Ekanayake et al) discloses tea extract having reduced bitter and astringent flavors and low levels of polymerized or oxidized flavanol. The extract is produced by separating an acidified solution containing added protein. In accordance with U.S. Pat. No. 5,879,733, issued to Ekanayake et al on Mar. 9, 1999, a green tea extract having improved clarity and color is produced by combining cation exchange resin and nanofiltration in the extraction process.

All of the thus generated herbal extracts can be subjected to further concentration through evaporation or nano or ultra filtration to form concentrated liquid herbal extracts, or to spray drying or freeze drying to form more stable dried powders. For a complete review of tea processing which can, in many cases, be applied to other plants, reference is made to the latest edition of Kirk-Othmer Encyclopedia of Chemistry published by John Wiley & Son.

When considering the increasing importance of plants and herbal extracts as functional and therapeutic agents, there are limits to known processing methods. The limits are related to the efficiency of the extraction process and to the bioavailability of the active ingredients.

Bioavailability of active ingredients is a prerequisite for achieving optimum effects. The bioavailability of a substance determines the ability of a person to utilize the substance. Increased bioavailability results in a high percentage of the substance being utilized instead of being eliminated. As a matter of fact, a small amount of a substance having good bioavailability provides much more benefit and effect than a larger amount of the same substance in a poor bioavailability form. As well as the chemical state of the active ingredient and the composition of the substances that come with it, many factors can affect bioavailability. U.S. Pat. No. 6,001,393 (Daoud), issued on Dec. 14, 1999 describes the making of a ginkgo biloba extract with enhanced bioavailability by post extraction addition of polyols such as xylitol, maltitol, mannitol and others. It is well known that ingestion of certain oligosaccharides, disaccharides and polyols promotes the growth of beneficial gastrointestinal tract bacteria which in turn stimulates gastrointestinal absorption. Indigestible oligosaccharides and slowly absorbable disaccharides and polyols provide fermentable substrates for lactic acid bacteria in the colon. Such substances are referred to as “prebiotic” and the concept was first introduced by Gibson and Roberfroid in 1994 (Gibson, G. R., and Roberfroid, M. Dietary modulation of human colonic microbiota—introducing the concept of prebiotic. J. Nutr. 1995; 125: 1401-1412).

Probiotic bacteria are also known to promote gastrointestinal absorption. U.S. Pat. No. 6,080,401, issued to Reddy et al on Jul. 27, 2000 teaches that the curative action of herbal remedies and pharmaceutical drugs is enhanced and accelerated by administering such drugs in combination with prebiotic bacteria. The curative effect of the drugs is enhanced and accelerated without creating harmful side effects.

Thus, when mixed with curative plants and/or herbal extracts, prebiotics and probiotics can be used separately or in combination to increase bioavailability.

The literature does not appear to describe the direct anaerobic fermentation of plants and herbal extracts using probiotic bacteria. The term “fermented” is occasionally used in the art to describe the oxidation process of plant leaves or other parts by endogenous enzymes. For example, U.S. Pat. No. 5,863,681, issued to Barrett et al on Jan. 26, 1999 clearly describes a tea leaf fermentation process as being an “enzymatic oxidation” process. The Hagiwara Canadian Patent Application 2,176,293 provides examples of enzymes responsible for the blackening, discoloration and deterioration of green plants, namely chlorophyllases, peroxidases and polyphenol oxidases.

It is well known that some food can be fermented to provide enhanced flavor, modified texture, physiological feedback and/or added nutritional value. In the case of physiological feedback and added nutritional value, a food stuff is referred as a “functional” food. An example of a functional food is provided by U.S. Pat. No. 5,891,492 issued to Ishigaki on Apr. 6, 1999. The inventor describes a fermentation product of sesame having antioxidative properties. The fermentation process is performed by seeding a sesame product with lactic acid bacteria and by adding a nutrition source which supports and enhances fermentation.

### GENERAL DESCRIPTION OF THE INVENTION

The object of the present invention is to provide a novel method for extracting therapeutically and/or physiologically active ingredients from plants which increases the bioavailability of the ingredients by the direct fermentation of plant parts using probiotic bacteria.

Accordingly, the invention relates to a process for preparing a herbal extract comprising the steps of:

(a) mixing herbal matter with water to produce an aqueous extract solution,

(b) adding a nutritive supplement capable of supporting bacterial fermentation to the solution,

(c) seeding the resulting mixture with a probiotic bacteria, and

(d) incubating the seeded mixture to effect fermentation of the herbal matter.

### DESCRIPTION OF THE PREFERRED EMBODIMENT

As used herein, by “herb” is meant any photosynthetic vascular organism, with no further distinction between plants, flowers, herbs, weeds, trees or other commonly used term.

As used herein, by “phytotherapeutic” is meant any herb combination which is pharmaceutically acceptable and which has a curative effect.

As used herein, “functional” means any herb combination, which is pharmaceutically acceptable, and which can provide physiological feedback.

As used herein, by “prebiotic” is meant any nondigestible food ingredient which beneficially affects the host by selectively stimulating the growth and/or the activity of one or a limited number of bacteria in the colon.

As used herein, the term “probiotic” means any combination of live microorganisms which upon ingestion beneficially affect the host by improving intestinal microbial balance.

As used herein, the term “sp” means any species, subspecies, serotype, serogroup and biovar that can be included in a selected gender.

Strength of the composition: all of the concentration limits of components are single strengths.

All amounts, parts, ratios and percentages used herein are in weight by weight unless otherwise stated. All weights or herbal ingredients are provided on the basis of dry weight.

Starting Material and Pre-Treatment Steps.

The first part of the process is to make a liquid herbal formulation suitable for fermentation. Any phytotherapeutic and/or functional herb combination can be used as the starting material. Freshly gathered, partly oxidized or dried herbs in the form of entire parts or crushed powder can be used. Leaves, flowers, stems, seeds, barks, roots or any other parts of herbs are suitable for use. Any commercially available herbal extract can also be used. Herbal extracts are usually available in concentrated liquid or dried powder form. For the purpose of the invention, the preferred form of starting material is dried herbal powders which have not been subjected to enzymatic oxidation prior to drying.

The herbal material is added to cold water, which is rapidly brought to a boil. The concentration of the herbal material is 0.01 to 15%, preferably 0.1 to 10% and optimally 1 to 6%. A nutritive supplement to support the bacterial fermentation process is added to the solution. Any carbon and nitrogen source can be used. The nutritive supplement carbon source is selected from sucrose, dextrose, maltose, fructose, starch and blackstrap molasses at a concentration of 0.001 to 10%, and preferably 0.1 to 1% of the solution. Any other fermentable carbon source such as a monosaccharide, a disaccharide or an oligosaccharide can be used. The nitrogen source is selected from yeast extract, corn steep and meat and plant hydrolyzed extracts such as peptone from pancreatically digested casein, soytone and peptone at a concentration of 0.001 to 10% and preferably 0.01 to 1% of the solution. The preferred composition of the supplement includes dextrose at a concentration of 0.05 to 0.5%, blackstrap molasses at a concentration of 0.02 to 0.1%, yeast extract at a concentration of 0.05 to 0.5% and peptone from pancreatically digested casein at a concentration of 0.005 to 0.05%.

The herbal solution containing the nutritive supplement is boiled for a minimum of 15 minutes and then transferred to bioreactor for heat sterilization. The temperature is ramped to 121° C. at 15 psi for 30 minutes. The solution is then cooled down under pure filter sterilized nitrogen pressure as rapidly as possible to 37° C. L-cysteine is filter sterilized on a 0.2 micrometer pore size filter and aseptically added to the herbal solution as an oxygen scavenger at a final concentration of 0.01 to 0.15% of the solution. Other oxygen scavengers can be used, for example ascorbic acid at a final concentration of 0.01 to 0.15%.

The Fermentation Process

The fermentation process is performed using any suitable probiotic bacteria able to grow and perform a prolonged heterofermentation or homofermentation under the defined conditions. The fermentation process can be performed using single bacterial strains or mixed bacterial populations. The probiotic bacteria used to perform the fermentation process are preferably selected from the following groups and genders of bacteria. Among the lactic acid bacteria group any Aerococcus sp, Alloiococcus sp, Carnobacterium sp, Dolosigranulum sp, Enterococcus sp, Globicatella sp, Lactobacillus sp, Lactoccoccus sp, Lactosphaera sp, Leuconostoc sp, Oenococcus sp, Pediococcus sp, Streptococcus sp, Tetragenococcus sp, Vagococcus sp and Weissella sp. Even Bifidobacterium sp, which are not strictly lactic acid bacteria and are phylogenetically unrelated and have a distinct mode of sugar fermentation are very good candidates for use in the method of the present invention. Any Bifidobacterium sp can be used in the fermentation process. Propionobacterium sp can also used in the fermentation process. In addition, any lactic acid producing bacteria such as Bacillus sp, Paenibacillus sp and Brevibacillus sp can be used in the fermentation process.

The selected bacteria must be nonpathogenic, i.e. safe for human consumption and preferably exhibit one or more probiotic properties, namely acid and bile stability, bile deconjugation, adherence to human intestinal mucosa, colonization of the human gastrointestinal tract, production of antimicrobial substances, antagonism against cariogenic and pathogenic bacteria or any other property that can be beneficial to human health.

The preferred bacterial strains are Lactobacillus acidophilus, Lactobacillus casei and Lactobacillus rhamnosus. They are preferably used in a mixed culture during the fermentation process.

A bioreactor containing the heat sterilized starting material, the nutritive supplement and the oxygen scavenger, is seeded with a late logarithmic population of bacteria. The seeding bacteria are grown in any nutritive medium which can support their growth. The preferred growth medium has the composition set out in Table 1.

TABLE 1

| | Concentration |
|---|---|
| Ingredient | (g/L) |
| Yeast extract | 15 |
| Peptone from pancreatically | 1 |
| digested casein | |
| Dipotassium phosphate | 2 |
| Dextrose | 15 |
| Blackstrap molasses | 5 |
| Magnesium sulfate | 0.2 |
| Sodium acetate | 5 |
| L-cysteine | 0.5 |
| Manganese sulfate | 0.04 |
| Balance water | |

The pH of the medium is adjusted to 6.8 and heat sterilized at 121° C. and 15 psi for 20 minutes. Separate vessels containing the seeding growth medium are seeded with single pure strains of bacteria. The media are incubated at 37° C. for a period sufficient to reach late logarithmic growth stage. The seeding cultures are aseptically added to the contents of the bioreactor. The volume of the seeding culture is 0.01 to 10% of the volume of the starting material in the bioreactor, preferably 0.1 to 5% and optimally 1 to 3%.

The fermentation process is performed best by maintaining the pH of the culture at 6.8 for a minimum of 6-8 hours using an inorganic base such as sodium hydroxide or potassium hydroxide and preferably ammonium hydroxide. Any other inorganic or organic base which is not deleterious to the bacterial population and which is acceptable in human nutrition can be used. After 6-8 hours, the bacterial culture has normally reached a very active growing and metabolic stage. The pH control is then interrupted to allow acidification of the medium which will be partly responsible for the stability of the end product and for more complete extraction of the herbal components.

The fermentation process is stopped when no further pH reduction and no optical density increase at 660 nm is recorded for a minimum of two consecutive hours.

Preparation and Stabilization of the Commercial Formulation

The best phytotherapeutic, probiotic and functional properties are provided when the fermented herbal extract is dried. Drying also provides the best stabilization conditions. Suitable methods of drying include spray drying, freeze drying and sprouted bed drying. The fermented herbal bacteria can be directly spray dried but the inventors have noted that the best survival rate of the probiotic population is achieved when the bacteria are separated from the fermented herbal extract and separately freeze dried. In this process, the bacterial population and the residual herbal solid particles are concentrated by centrifuging or preferably by tangential flow filtration using cassettes or hollow fiber cartridges. If the starting material contains entire or large herb parts, it is necessary to press the herbal material to separate the fermented extract from the herb debris prior to tangential flow filtration. If the herbal solid residue includes particles bigger than 1 mm, a gross filtration step should be performed to remove such particles prior to tangential flow filtration. If centrifugation is used, the gross filtration step may not be necessary. The pH of the bacterial concentrate is adjusted to 6-8, preferably 6.5-7.5 with any inorganic acid or base, and a suitable cryoprotectant is added prior to freeze drying. Cryoprotectants for freeze drying of bacterial culture are well known in the art, but the preferred cryoprotection is provided by adding sucrose at a concentration of 1 to 5%, malto-dextrin at a concentration of 1 to 5% and glycerol at a concentration of 0.1 to 0.5%. The bacterial culture is then freeze dried using standard freeze drying conditions. In the preferred process, the bacterial free herbal extract is separately dried using an available drying method. For example, in the case of spray drying, hot spray drying using hot air at 140 to 170° C. or cold spray drying using air dried with a suitable desiccant may be employed.

After being separately dried, the probiotic bacteria and the fermented herbal extract powders can be mixed together. The dry formulation can be further supplemented with any ingredient that can contribute to the targeted health effects. It can be beneficial to add prebiotic substances such as selectively fermentable monosaccharides, disaccharides and oligosaccharides and short chain fatty acids such as propionic and butyric acids. The prebiotic substances can contribute to the targeted health or functional effect by increasing the bioavailability of the active ingredients and by stimulating the beneficial indigenous gastrointestinal bacterial populations than can promote intestinal absorption. Examples of prebiotic substances are soy-oligosaccharides, xylo-oligosaccharides, galacto-oligosaccharides, fructo-oligosaccharides, isomalto-oligosaccharides, lacto-fructo-oligosaccharides (lactosucrose), lactulose, palantinose, lactitol, xylitol, sorbitol and mannitol. The substances are used at concentrations of 5 to 90%, preferably 25 to 85% and optimally 60 to 80% of the dried formulation.

It is also advisable to add an appropriate carrier to help the probiotic bacteria survive passage through the harsh conditions of the stomach. The preferred carriers are high amylase starch as described in U.S. Pat. No. 6,060,050, issued to Brown et al on May 9, 2000, and shortening with a melting point higher than 30° C. as described in the Canadian Patent Application No. 2,292,325, filed on Dec. 13, 1999. Any other carriers that can protect the probiotic bacteria during passage through the stomach can also be used. Carriers are used in concentrations of 5 to 90%, preferably 25 to 85% and optimally 60 to 80% of the dried formulation.

Formulations of the present invention may be in the form of capsules, cachets or tablets, powder, granules or micro-encapsulated granules.

In some cases, the fermented herbal extract could be delivered as a liquid formulation. The inventors have noted that, compared to dried formulations, a liquid formulation can provide most of the beneficial phytotherapeutic and functional effects with the major difference that the probiotic effects cannot be maintained, because the groups of bacteria used in this invention are highly unstable in the liquid phase. The survival rate of the bacteria is almost negligible after one week storage at room temperature. This drawback can be circumvented if sporulating bacteria are used to ferment the herbal extract. Fermenting, non-pathogenic probiotic Bacillus sp, Paenibacillus sp and Brevibacillus sp could be used to maintain the probiotic effect in liquid formulations. In the case of a liquid formulation, the fermented herbal extract would have to be stabilized and protected against bacterial and fungal deterioration.

The first step is to pasteurize the fermented herb extract to ensure a controlled and immediate mortality of the probiotic population. Organic acids are then added to either acidify the formulation or to provide bacterial and fungal growth inhibition. Any edible organic acids such as ascorbic, erythorbic, fumaric, citric, malic, acetic, caprylic, lactic, propionic, adipic, tartaric and succinic can be used. If necessary, an inorganic acid can be used to lower the pH. Suitable inorganic acids include phosphoric and carbonic acids. The pH of the liquid formulation is 2-4 and preferably 3.0-3.8. The use of the listed organic acids usually provides sufficient stability against most bacterial deteriorating agents, but in some cases it might be preferable to use food preservatives to prevent fungal deterioration, thereby ensuring long shelf life. Such preservatives are abundant and well known in the art, and include sodium and potassium benzoate, sodium and potassium sorbate, sodium and calcium propionate and other food grade preservative produced by culture of Propionobacterium. Natamycin, a fungal inhibiting substance produced by the culture of Streptomyces nataliensis, can also be used. Natamycin is produced by Gist Brocades and is available on the market under the trademark mark of Delvocid. The preferred preservative is citric acid in combination with ascorbic acid, benzoic acid and sorbic acid at concentrations of 0.1 to 2%, 0.1 to 1%, 0.03 to 0.1% and 0.03 to 0.1%, respectively. Longer shelf life stability can be achieved by cold temperature preservation.

As in the case of the dry formulation, the liquid formulation can be supplemented with prebiotic substances. The prebiotics can partly compensate for the lack of beneficial probiotic effects caused by the killing of the bacterial population. In the case of the liquid formulation, the concentration of the prebiotic substances is 5 to 40%, preferably 10 to 30% and optimally 15 to 20% of the liquid formulation weight.

Although it is not necessary for achieving the phytotherapeutic and functional effects, many ingredients such as colorants, sweeteners, flavors and thickeners can be added to improve the appearance and taste of the formulation.

Many food grade thickeners are available on the market but the preferred thickening agent is xanthan gum used at a concentration of 0.005 to 0.08% and preferably 0.01 to 0.04%.

Any natural or artificial food grade colorant can be added to the liquid formulation.

Any sweeteners such as sugars, sugar alcohols and non-caloric sweeteners can be added to the liquid formulation to provide a more pleasant taste. For sugars and sugar alcohols, the amount of sweetener can be 1 to 15%, preferably 5 to 15% and optimally 9 to 12%. Some of the sweetening taste can be provided by some prebiotics added to the liquid formulation as described above.

Flavors which can be added to the liquid formulation can be natural or artificial fruit or botanical flavors. In the case of flavor addition, it may be necessary to add food grade weighting agents or emulsifiers to avoid phase separation of the flavor.

The invention is described in even greater detail in the following examples.

### EXAMPLES

### Example 1

Green tea powder, gunpowder tea powder, ground ivy (Glechoma hederacea) leaves powder, yeast extract, peptone from pancreatically digested casein, dextrose and blackstrap molasses are added to a small amount of fresh water. The liquid and additives are thoroughly mixed, and sufficient water is added to bring the volume to 8 liters.

The final concentration of the ingredients are set out in Table 2.

TABLE 2

| | Concentration |
|---|---|
| Ingredient | (g/L) |
| Green tea powder | 20 |
| Gunpowder tea powder | 20 |
| Ground Ivy (Glechoma | 20 |
| Hederacea) | |
| Yeast extract | 1.87 |
| Peptone from | 0.125 |
| Pancreatically digested | |
| Casein | |
| Dextrose | 1.87 |
| Blackstrap molasses | 0.625 |

The mixture is brought to a boil for 15 minutes and then transferred to a ten iter New Brunswick BioFlow-3 bioreactor. The temperature is ramped to 121° C. at 15 psi for 30 minutes. The solution is then cooled under pure filter sterilized nitrogen pressure as rapidly as possible to 37° C. L-cysteine is filtered sterilized on a 0.2 micrometer pore size filter and aseptically added to the herbal solution as an oxygen scavenger at a final concentration of 0.5 g/liter of solution.

One strain of Lactobacillus acidophilus, one strain of Lactobacillus casei and one strain of Lactobacillus rhamnosus are used to seed the bioreactor. Those strains are of human origin and present the phenotypes listed in Table 3.

TABLE 3

| | L. acidophilus | L. rhamnosus | L. casei |
|---|---|---|---|
| Substrate | (BlbC) | (BlbD) | (BlbB) |
| Glycerol | − | − | − |
| Erythritol | − | − | − |
| D-Arabinose | − | − | − |
| L-Arabinose | − | − | − |
| Ribose | − | + | − |
| D-Xylose | − | − | − |
| L-Xylose | − | − | − |
| Adonitol | − | − | − |
| β-Methyl-xyloside | − | − | − |
| Galactose | + | + | + |
| D-Glucose | + | + | + |
| D-Fructose | + | + | + |
| D-Mannose | + | + | + |
| 1-Sorbose | − | + | − |
| Rhamnose | − | −+ | − |
| Dulcitol | − | − | − |
| Inositol | − | − | − |
| Mannitol | − | + | + |
| Sorbitol | − | + | − |
| α-Methyl-D-mannoside | − | − | − |
| α-Methyl-D-glucoside | −+ | + | − |
| N-Acetyl glucosamine | + | + | + |
| Amygdalin | + | + | + |
| Arbutin | + | + | + |
| Esculin | − | + | + |
| Salicin | + | + | + |
| Cellobiose | + | + | + |
| Maltose | + | −+ | − |
| Lactose | + | + | + |
| Melibiose | + | − | − |
| Saccharose | + | − | + |
| Trehalose | − | + | + |
| Inulin | − | − | − |
| Melezitose | − | + | + |
| D-Raffinose | + | − | − |
| Amidon | + | − | − |
| Glycogen | − | − | − |
| Xylitol | − | − | − |
| β-Gentiobiose | + | − | + |
| D-Turanose | − | + | − |
| D-Lyxose | − | − | − |
| D-Tagatose | + | + | + |
| D-Fucose | − | − | − |
| L-Fucose | − | − | − |
| D-Arabitol | − | − | − |
| L-Arabitol | − | − | − |
| Gluconate | − | − | + |
| 2-ceto-gluconate | − | − | − |
| 5-ceto-gluconate | − | − | − |

The strains are seeded as late logarithmic populations separately grown in 100 ml of a culture media placed in a closed and filled bottle kept at 37° C. for 16 hours without shaking. The culture media has the composition set out in Table 1.

After seeding with the three bacterial strains, the pH is adjusted to 6.8 using ammonium hydroxide. The general fermentation conditions are slight mixing at 100 rpm, temperature maintained at 37° C. and no aeration. Using a pH controller, the pH is automatically kept at 6.8 during the first six hours of the fermentation process. Ammonium hydroxide is used to maintain the pH.

After 6 hours, the pH controller is stopped to allow acidification of the media which will be partly responsible for the stability of the end product and for more complete extraction of the herbal components.

The fermentation process is stopped when no further pH reduction and no optical density increase at 660 nm are recorded for a minimum of two consecutive hours. For the composition and conditions set out above the average fermentation time is 16 hours.

The fermented herbal extract is filtered on a filter which removes all solid particles bigger than 0.1 mm. The filtered solution is then subjected to tangential flow microfiltration using a laboratory scale KOCK MEMBRANE™ hollow fiber cartridge with a molecular weight cut-off of 500,000. The bacterial mass is concentrated by a factor of 15 relative to the starting volume and the permeate is kept as the fermented herbal solution.

The pH of the bacterial concentrate is adjusted to approximately 7.0 with sulphuric acid or sodium hydroxide, and sucrose (50 g/liter), malto-dextrin (50 g/liter) and glycerol (5 g/liter) are added as cryoprotectants. The bacterial concentrate is frozen in a shallow tray at −20° C. and freeze dried for 24 hours.

The fermented herbal extract is spray dried with hot air at 150° C. using a Buchi mini spray drier model B-191.

The entire bacterial powder is mixed with the entire fermented herbal extract powder. Fructo-oligosaccharide is mixed with the powder as a prebiotic to make up 80% of the total weight of the preparation. The preparation is encapsulated and kept in a sealed jar containing a desiccating pouch.

This preparation is used as a metabolic stimulant. It has functional properties in providing physiological awareness as well as being a powerful anti-oxidant. For optimal functional effects, it is recommended to take three capsules one hour after breakfast and one hour after lunch.

### Example 2

The following describes the preparation of a dried, phytotherapeutic, fermented herbal extract that has enhanced physiological and probiotic effects.

Entire plant artichoke powder, dandelion (Taraxacum officinale) root powder, strawberry leaf powder, yeast extract, peptone from pancreatically digested casein, dextrose and blackstrap molasses are thoroughly mixed with a small volume of fresh water. Additional water is added to bring the volume to 8 liters.

The final concentrations of the ingredients are listed in Table 4.

TABLE 4

| | Concentration |
|---|---|
| Ingredient | (g/L) |
| Artichoke powder | 20 |
| Dandelion (Taraxacum officinale) | 20 |
| Root powder | |
| Strawberry leaf powder | 20 |
| Yeast extract | 1.87 |
| Peptone from pancreatically | 0.125 |
| Digested casein | |
| Dextrose | 1.87 |
| Blackstrap molasses | 0.625 |

The mixture is brought to a boil for 15 minutes and then transferred to a ten liter New Brunswick BioFlow-3 bioreactor. The temperature is ramped to 121° C. at 15 psi for 30 minutes. The solution is then cooled under pure filter sterilized nitrogen pressure as rapidly as possible to 37° C. L-cysteine is filter sterilized on a 0.2 micrometer pore size filter and aseptically added to the herbal solution as an oxygen scavenger at a final concentration of 0.5 g/liter.

One strain of Lactobacillus acidophilus, one strain of Lactobacillus casei and one strain of Lactobacillus rhamnosus are used to seed the bioreactor. The strains are the same as described in the Example 1.

The strains are seeded as late logarithmic populations separately grown in 100 ml of a culture medium placed in a closed and filled bottle, which is kept at 37° C. for 16 hours without shaking. The culture medium has the composition set out in Table 1.

After seeding the three bacterial strains, the pH is adjusted to 6.8 with ammonium hydroxide. The general fermentation conditions are slight mixing at 100 rpm, temperature maintained at 37° C. and no aeration.

Using a pH controller, the pH is automatically kept at 6.8 during the first six hours of the fermentation process. Ammonium hydroxide is used to maintain the pH.

After this period the pH controller is stopped to allow acidification of the medium which will be partly responsible for the stability of the end product and for more complete extraction of the herbal components.

The fermentation process is stopped when no further pH reduction and no optical density increase at 660 nm are recorded for a minimum of two consecutive hours. For the described composition and conditions the average fermentation time is 16 hours.

The fermented herbal extract is filtered on a filter which removes all solid particles bigger than 0.1 mm. The filtered solution is then subjected to tangential flow microfiltration using a laboratory scale KOCH MEMBRANE™ hollow fiber cartridge with a molecular weight cut-off of 500,000. The bacterial mass is concentrated by a factor of 15 relative to the starting volume and the permeate is kept as the fermented herbal solution.

The pH of the bacterial concentrate is adjusted to approximately 7.0 with sulphuric acid or sodium hydroxide, and sucrose (50 g/liter), malto-dextrin (50 g/liter) and glycerol (5 g/liter) are added as cryoprotectants. The bacterial concentrate is frozen in a shallow tray at −20° C. and freeze dried for 24 hours.

The fermented herbal extract is spray dried with hot air at 150° C. using a Buchi mini spray drier model B-191.

The entire bacterial powder is mixed with the entire fermented herbal extract powder. Fructo-oligosaccharide is mixed with the powders as a prebiotic to total 80% of the total weight of the preparation. The preparation is encapsulated and kept in a sealed jar containing a desiccating pouch.

The preparation is used as a hepatic stimulant. The preparation has phytotherapeutic properties in stimulating bile secretion and liver functions. For optimal functional effects, it is recommended to take three capsules immediately after a meal.

### Example 3

The following describes the preparation of a liquid functional fermented herbal extract that has enhanced physiological effects.

Green tea powder, gunpowder tea powder, ground ivy (Glechoma hederacea) leaf powder, yeast extract, peptone from pancreatically digested casein, dextrose and blackstrap molasses are added to a small volume of fresh water. The composition is thoroughly mixed and water is added to bring the volume to 8 liters.

The final concentrations of the ingredients are set out in Table 5.

TABLE 5

| | Concentration |
|---|---|
| Ingredient | (g/L) |
| Green tea powder | 20 |
| Gunpowder tea powder | 20 |
| Ground Ivy (Glechoma hederacea) | 20 |
| Leaf powder | |
| Yeast extract | 1.87 |
| Peptone from pancreatically | 0.125 |
| Digested casein | |
| Dextrose | 1.87 |
| Blackstrap molasses | 0.625 |

The mixture is brought to a boil for 15 minutes and then transferred to a ten liter New Brunswick BioFlow-3 bioreactor. The temperature is ramped to 121° C. at 15 psi for 30 minutes. The solution is then cooled down under pure filter sterilized nitrogen pressure as rapidly as possible to 37° C. L-cysteine is filter sterilized on 0.2 micrometer pore size filter and aseptically added to the herbal solution as an oxygen scavenger at a final concentration of 0.5 g/liter.

One strain of Lactobacillus acidophilus, one strain of Lactobacillus casei and one strain of Lactobacillus rhamnosus are used to seed the bioreactor. Those strains are the same as described in Example 1.

The strains are seeded as late logarithmic populations separately grown in 100 ml of a culture media placed in a closed and filled bottle that is kept at 37° C. for 16 hours without shaking. This culture media has the composition set out in Table 1.

After seeding the three bacterial strains, the pH is adjusted to 6.8 with ammonium hydroxide. The general fermentation conditions are slight mixing at 100 rpm, temperature maintained at 37° and no aeration.

Using a pH controller, the pH is automatically kept at 6.8 during the first six hours of the fermentation process. Ammonium hydroxide is used to maintain the pH. After this period, the pH controller is stopped to allow acidification of the media which will be partly responsible for the stability of the end product and for more complete extraction of the herbal components.

The fermentation process is stopped when no further pH reduction and no optical density increase at 660 nm are recorded for a minimum of two consecutive hours. Using the above described composition and conditions, the average fermentation time is 16 hours.

The fermented herbal extract is filtered on a filter which removes all solid particles bigger than 0.1 mm. Ascorbic acid is added to a final concentration of 0.5%, citric acid to a final concentration of 0.1%, sodium benzoate to a final concentration of 0.075% and potassium sorbate to a final concentration of 0.075%. A water soluble natural flavor of orange-lemon taste is added at a final concentration of 0.1%. The pH is adjusted to 3.0 using 85% phosphoric acid.

The fermented herbal extract is pasteurized and kept at room temperature in bottles protecting the composition from light.

The preparation is used as a metabolic stimulant. The preparation has functional properties in providing physiological awareness as well as being a powerful anti-oxidant For optimal functional effects, it is recommended to add 10 ml of the composition to 125 ml of hot water, juice or fresh water and to drink the liquid one hour after breakfast and one hour after lunch.

### Example 4

Entire plant artichoke powder, dandelion (Taraxacum officinale) root powder, strawberry leaf powder, yeast extract, peptone from pancreatically digested casein, dextrose and blackstrap molasses are added to a small volume of fresh water. The composition is thoroughly mixed, and water is added to bring the volume to 8 liters.

The final concentrations of the ingredients are set out in Table 6 below.

TABLE 6

| | Concentration |
|---|---|
| Ingredient | (g/L) |
| Artichoke powder | 20 |
| Dandelion (Taraxacum officinale) | 20 |
| Root powder | |
| Strawberry leaf powder | 20 |
| Yeast extract | 1.87 |
| Peptone from pancreatically | 0.125 |
| Digested casein | |
| Dextrose | 1.87 |
| Blackstrap molasses | 0.625 |

The composition is brought to a boil for 15 minutes and then transferred to a ten liter New Brunswick BioFlow-3 bioreactor. The temperature is ramped to 121° C. at 15 psi for 30 minutes. The solution is then cooled down under pure filter sterilized nitrogen pressure as rapidly as possible to 37° C. L-cysteine is filter sterilized on a 0.2 micrometer pore size filter and aseptically added to the herbal solution as an oxygen scavenger at the final concentration of 0.5 g/liter.

One strain of Lactobacillus acidophilus, one strain of Lactobacillus casei and one strain of Lactobacillus rhamnosus are used to seed the bioreactor. The strains are the same as described in the Example 1.

The strains are seeded as late logarithmic populations separately grown in 100 ml of a culture medium placed in a closed and filled bottle, which is kept at 37° C. for 16 hours without shaking. The culture medium has the composition set out in Table 1.

After seeding the three bacterial strains, the pH is adjusted to 6.8 with ammonium hydroxide. The general fermentation conditions are slight mixing at 100 rpm, temperature maintained at 37° C. and no aeration.

Using a pH controller, the pH is automatically kept at 6.8 during the first six hours of the fermentation process. Ammonium hydroxide is used to maintain the pH.

After this period the pH controller is stopped to allow acidification of the medium which will be partly responsible for the stability of the end product and for more complete extraction of the herbal components.

The fermentation process is stopped when no further pH reduction and no optical density increase at 660 nm are recorded for a minimum of two consecutive hours. For the described composition and conditions the average fermentation time is 16 hours.

The fermented herbal extract is filtered on a filter which removes all solid particles bigger than 0.1 mm. Ascorbic acid is added to a final concentration of 0.5%, citric acid to a final concentration 0.1%, sodium benzoate to a final concentration 0.075% and potassium sorbate to a final concentration 0.075%. A water soluble natural flavor of orange-lemon taste is added at a final concentration of 0.1%. The pH is adjusted to 3.0 using 85% phosphoric acid solution.

The fermented herbal extract is pasteurized and kept at room temperature in bottles protecting the composition from light.

The preparation is used as a hepatic stimulant. It has phytotherapeutic properties in stimulating bile secretion and liver functions. For optimal phytotherapeutic effects, it is recommended to add 10 ml of the composition to 125 ml of hot water and to drink it after each meal.

We claim:

1. A process for preparing a herbal extract comprising the steps of:
(a) mixing herbal matter with water to produce an aqueous extract solution,
(b) adding a nutritive supplement capable of supporting bacterial fermentation to the solution,
(c) seeding the resulting mixture with probiotic bacteria, and
(d) incubating the seeded mixture to effect fermentation of the herbal matter.

2. A process according to claim 1, wherein the nutritive supplement includes a fermentable carbon source selected from the group consisting of a monosaccharide, a disaccharide and an oligosaccharide; and a nitrogen source selected from the group consisting of yeast extract, corn steep, and meat and plant hydrolyzed extracts.

3. A process according to claim 2, wherein the carbon source is selected from the group consisting of sucrose, maltose, dextrose, fructose, starch and blackstrap molasses at a concentration of 0.001 to 10% of the extract solution; and the nitrogen source is selected from the group consisting of peptone from pancreatically digested casein, soytone and peptone at a concentration of 0.001 to 10% of the extract solution.

4. A process according to claim 1, wherein the nutritive supplement comprises yeast extract, peptone from pancreatically digested casein, dextrose, blackstrap molasses, and L-cysteine in water.

5. A process according to claim 1, wherein the probiotic bacteria is a lactic acid bacteria selected from the group consisting of Aerococcus sp, Alloiococcus sp, Carnobacterium sp, Dolosigranulum sp, Enterococcus sp, Globicatella sp, Lactobacillus sp, Lactccoccus sp, Lactosphaera sp, Leuconostoc sp, Oenococcus sp, Pediococcus sp, Streptococcus sp, Tetragenococcus sp, Vagococcus sp and Weisella sp, and Bifidobacterium sp and Propionobacterium sp or selected from the lactic acid producing Bacillus sp, Brevibacillus sp and Paenibacillus sp.

6. A process according to claim 1, wherein the probiotic bacteria is selected form the group consisting of Lactobacillus acidophilus, Lactobacillus casei and Lactobacillus rhamnosus.

7. A process according to claim 1, wherein the mixture is dried following fermentation to yield a dry formulation of herbal extract.

8. A process according to claim 7, wherein the mixture is freeze dried in the presence of a cryoprotectant.

9. A process according to claim 8, wherein the cryoprotectant is sucrose, malto-dextrin and glycerol.

10. A process according to claim 7, wherein a prebiotic substance is added to the dry formulation at a concentration of 5 to 90% by weight of the dry formulation.

11. A process according to claim 10, wherein the prebiotic substance is selected from the group consisting of soy-oligosaccharides, xylo-oligosaccharides, galacto-oligosaccharides, fructo-oligosaccharides, isomalto-oligosaccharides, lacto-fructo-oligosaccharides, lactulose, palantinose, lactitol, xylitol, sorbitol and mannitol.

12. A process according to claim 7, wherein a carrier is added to the dry formulation for protecting the probiotic bacteria during passage through a stomach.

13. A process according to claim 12, wherein the carrier is selected from a high amylase starch and shortening.

14. A process according to claim 1, wherein green tea powder, gunpowder tea powder, ground ivy leaves powder, yeast extract, peptone from pancreatically digested casein, dextrose and blackstrap molasses are mixed with water to produce a mixture; the mixture is boiled; L-cysteine is added to the boiled mixture as an oxygen scavenger; the mixture thus produced is heat sterilized and seeded with one strain of each of Lactobocillus acidophilus, Lactobacillus casei and Lactobacillus rhamnosus; and the seeded mixture is incubated to effect fermentation, thereby producing a fermented herbal extract for use as a metabolic stimulant.

15. A process according to claim 1, wherein entire plant artichoke powder, dandelion root powder, strawberry leaf powder, yeast extract, peptone from pancreatically digested casein, dextrose and blackstrap molasses are mixed with water to produce a mixture; the mixture is boiled; L-cysteine is added to the boiled mixture as an oxygen scavenger; the mixture thus produced is heat sterilized and seeded with one strain of each of Lactobocillus acidophilus, Lactobacillus casei and Lactobacillus rhamnosus, and the seeded mixture is incubated to effect fermentation, thereby producing a fermented herbal extract for use as a hepatic stimulant.

16. A process according to claim 1, wherein the mixture is maintained in a liquid state and stabilized using a food grade preservative

17. A process according to claim 16, wherein said preservative is an organic acid selected from the group consisting of ascorbic, erythorbic, fumaric, citric, malic, acetic, caprylic, lactic, propionic, adipic, tartaric and succinic acids.

18. A process according to claim 16, wherein said preservative is selected from the group consisting of sodium benzoate, potassium benzoate, sodium sorbate, potassium sorbate, sodium propionate, calcium propionate and natamycin.

19. A process according to claim 16, wherein the preservative is a combination of ascorbic acid, benzoic acid and sorbic acids at concentrations of 0.1 to 2%, 0.1 to 1% and 0.03 to 0.1%, respectively by volume of the aqueous herbal extract.
